(19) United States
(12) Plant Patent Application Publication
(10) Pub. No.: US 2016/0242333 P1
(43) Pub. Date: Aug. 18, 2016

(54) Mango tree named 'RA/17'

(50) ;

(71) Applicant: Kenneth Rayner, Katherine (AU)

(72) Inventor: Kenneth Rayner, Katherine (AU)

(21) Appl. No.: 14/544,769

(22) Filed: Feb. 13, 2015

(51) Int. Cl. A01H 5/00

(52) U.S. Cl. PLT/159

(57) ABSTRACT

A new mango tree named ‘RA/17’, distinguished by its attractive fruit having excellent flavor and a long shelf life.

### CROSS-REFERENCE TO RELATED APPLICATIONS

None

### GENUS AND SPECIES

Mangifera indica

### VARIETY DENOMINATION

‘RA/17’

### BACKGROUND AND SUMMARY OF THE VARIETY

The new mango tree ‘RA/17’ originated as a controlled cross of ‘Irwin’ (female parent, not patented) and ‘R2E2’ (male parent, not patented). The initial cross was made at Katherine, Northern Territory, Australia in 1996, and seedlings resulting from the cross were planted in 1997 in the same location. ‘RA/17’ was selected from among the seedlings for further observation due to its attractive fruit having excellent flavor and a long shelf life. In 2006, ‘RA/17’ was asexually propagated by grafting at Katherine, Northern Territory, Australia, and has since been observed to reproduce true to type over successive asexually propagated generations.

‘RA/17’ is distinguishable from its parents by the following characteristics:

| | ‘RA/17’ | ‘Irwin’ | ‘R2E2’ |
|---|---|---|---|
| Tree shape | Upright, open | Upright, semi-open | Upright, open |
| | canopy | canopy | canopy |
| Shape of dorsal | Rounded | Rounded | |
| shoulder in | outward | downward | |
| mature fruit | | | |
| Fruit size | Medium, | Small, | Large, |
| | 375 g to 560 g | 262 g | 600 to 1000 g |
| Fruit maturity | Early season | Mid-season | Mid- to late- |
| | | | season |

‘RA/17’ matures earlier than known varieties ‘Honey Gold’ (not patented) and ‘Van Dyke’ (not patented), both of which are considered late maturing varieties.

### BRIEF DESCRIPTION OF THE PHOTOGRAPHS

FIG. 1 shows fruit of the ‘RA17’ variety;

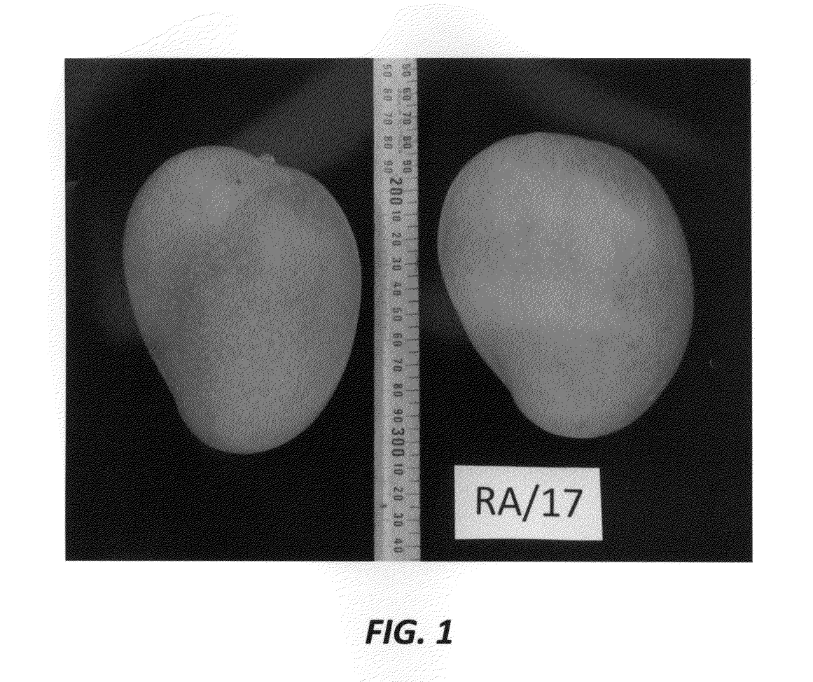

FIG. 2 shows fruit of the ‘RA17’ variety; and,

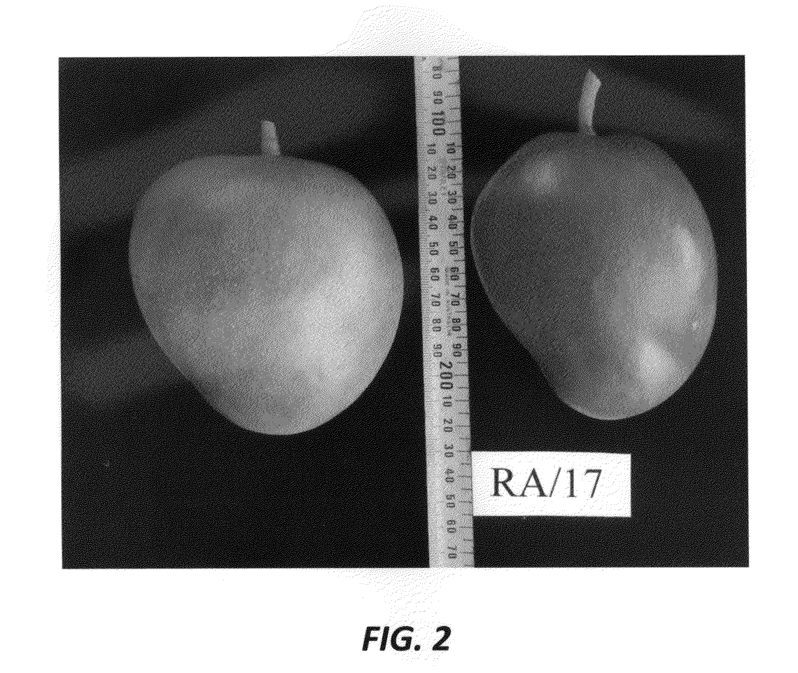

FIG. 3 shows a tree of the ‘RA17’ variety.

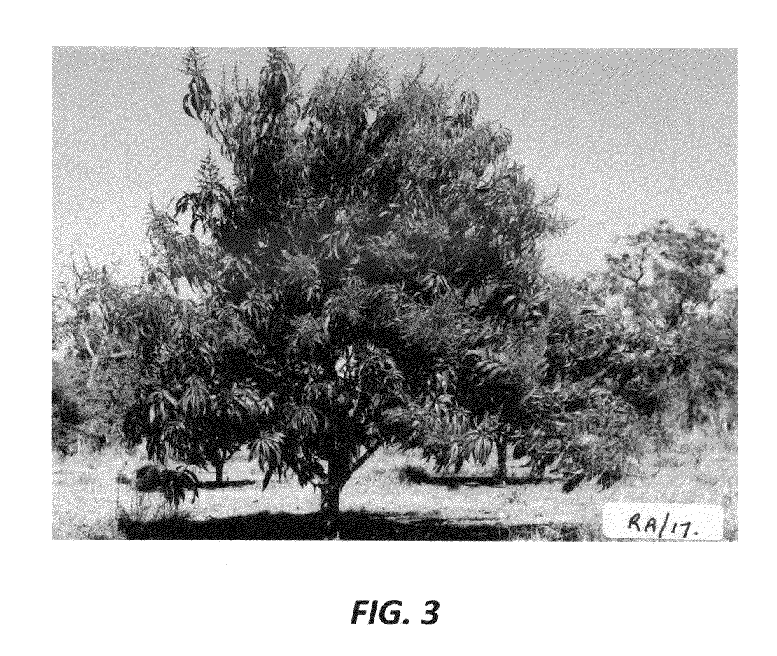

The colors of the claimed variety may vary with lighting conditions. Color characteristics of the variety should therefore be determined with reference to the observations described herein, rather than from these illustrations alone.

### DETAILED BOTANICAL DESCRIPTION

The following detailed botanical description is based on observations of trees planted in 2009 and grown on ‘Kensington Pride’ rootstock. Observations were recorded and photographs taken during the 2009 through 2012 growing seasons at Berry Springs, Northern Territory, Australia. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants of the new variety may vary from the stated average. Colors are described with reference to The Royal Horticultural Society Colour Chart (2005).
Tree:
   Vigor.—Vigorous.
   Height.—up to about 7.5 m.
   Spread.—up to about 6 m.
   Growth habit.—Narrow upright, open canopy.
   Productivity.—Good to very good; regular bearer.
   Trunk diameter.—29 cm.
   Bark texture.—Rough.
   Bark color.—Brown.
Branches:
   Bark color.—Old wood — brown; one-year-old wood — dark green.
   Bark texture.—Slightly rough.
Inflorescence:
   Number of years to first flowering.—Seedling — 5 years; Graft — 2 years.
   Regularity of flowering.—Very regular.
   Form.—Panicle.
   Inflorescence position.—Terminal.
   Axis growth habit.—Erect to drooping.
   Shape.—Broadly pyramidal.
   Length.—25 cm to 35 cm.
   Width.—25 to 30 cm.
   Color.—Red.
   Peduncle length.—6 mm.
   Pubescence.—None.
   Presence of leafy bracts.—Some seasons.
   Density of flowers.—Very dense.
   Type of flower.—Pentamerous.
   Stamen.—Usually one.
Leaves:
   Length.—28 cm.
   Width.—4.8 cm.
   Margin.—Entire, undulate.
   Pubescence.—None.
   Color — young leaf.—Upper surface — Green 136A.
   Color — mature leaf.—Upper surface — Green 137A Lower surface — Green 137C.
   Leaf shape.—Oblanceolate.
   Apex shape.—Acuminate.
   Base shape.—Cuneate.
   Petiole length.—5 cm.
   Petiole diameter.—3 mm.
   Petiole color.—Light green.
   Leaf orientation.—Petiole semi-erect, leaf drooping.
Fruit:
   Weight.—375 g to 560 g.
   Relative size.—Medium.
   Apex.—Obtuse.
   Form of shoulder.—Sloping abruptly.
   Form of stalk cavity.—Shallow.
   Stalk attachment.—Immature fruit — square on; mature fruit — recessed.
   Beak.—Not present.
   Skin.—Tough, thick.
   Skin color of immature fruit.—Green 131D; with direct sunlight, red 39B blush.
   Skin color of mature fruit.—Ground color orange 24A with red 41A blush.
   Flesh texture.—Very firm and juicy.
   Flesh color.—Orange 24A.
   Sweetness.—14 to 17 Brix.
   Fiber.—None.
   Aroma.—Mild, pleasant.
   Fruit per panicle.—1 to 4.
Stone:
   Length.—10 cm.
   Width.—5 cm.
   Depth.—4.8 cm.
   Venation.—Weak.
   Texture of stone fiber.—Fine.
   Adherence of fiber to stone.—Weak.
   Embryony.—Mono-embryonic.
   Storage.—Up to four weeks in a home refrigerator.
   Susceptibility to bruising.—Slight.
   Susceptibility to disease.—Slight.
   Relative harvest maturity.—Early.

I claim

1. a new and distinct variety of mango tree, substantially as illustrated and described herein.
